(19) United States
(12) Patent Application Publication
(10) Pub. No.: US 2024/0312625 A1
(43) Pub. Date: Sep. 19, 2024

(54) INTELLIGENT HEALTH ASSISTANT

(71) Applicant: Christiana Care Health System, Inc., Wilmington, DE (US)

(72) Inventors: Erwin Bautista, Aston, PA (US); Catherine Burch, Wilmington, DE (US); Phillip Chan, Wilmington, DE (US); Jonathan Meade, Wilmington, DE (US); Sarah Mooar, Wilmington, DE (US); Mustafa Mufti, Wilmington, DE (US); Kathryn Shirley, Wilmington, DE (US); John Evans, Landenberg, PA (US); Kelsey Kosinski, Wilmington, DE (US)

(21) Appl. No.: 18/606,906

(22) Filed: Mar. 15, 2024

Related U.S. Application Data
(60) Provisional application No. 63/452,294, filed on Mar. 15, 2023.

(51) Int. Cl. G16H 40/67; G16H 20/00; G16H 40/63

(52) U.S. Cl. CPC G16H 40/67; G16H 20/00; G16H 40/63

(57) ABSTRACT

An assistive device detects deterioration of a person's health, e.g., in an at-home setting, and offers guidance/prompting designed to address or avoid a deteriorated state, based on the person's personalized care plan. A deteriorated state is detected by analysis of multi-faceted data relating to any characteristics usable to assess the person's health, e.g., biometric, activity, or environmental data, as well as data from interactive health assessment screenings delivered via the assistive device. The care plan can contain actions identified by a clinician, e.g., as part of a post-discharge safety plan, or self-identified actions, which may be stabilizing coping mechanism activities. The device may also provide reminders of clinician visits, wellbeing/other activities, medications, and daily affirmations. The device may be configured as a mirror to display the person's image and an avatar with which the person may communicate in a dialog session to provide for a welcoming and engaging experience.

### CROSS-REFERENCE TO RELATED APPLICATION

This application claims the benefit of priority, under 35 U.S.C. § 119(e), of U.S. Provisional Patent Application No. 63/452,294, filed Mar. 15, 2023, the entire disclosure of which is hereby incorporated herein by reference.

### FIELD OF THE INVENTION

The present invention relates generally to systems and methods for monitoring for and recognizing changes in a person's health, and more particularly, to systems and methods for monitoring a person's health, such as mental health, and to provide help in accordance with the person's personalized care/safety plan.

### DISCUSSION OF RELATED ART

Persons with health conditions and/or issues may be closely monitored by healthcare providers, and be treated to provide care. For example, persons with mental health and/or behavioral health issues may be in need of psychiatric care and may be admitted for in-patent psychiatric hospitalization where the patient may be closely monitored by healthcare providers, and be treated to provide such care.

Subsequently, the patient is typically discharged and returned to an in-home setting, and thus no longer receives the same degree of close monitoring by on-site healthcare providers. While it may be possible to have a team of care providers manually and consistently/constantly monitoring discharged patients, or to have the patient responsible for consistently/constantly reporting to healthcare providers, such solutions are impractical, expensive and/or likely ineffective.

Notably, with respect to mental health, during the initial three months after discharge from an in-patient psychiatric hospitalization, patients are at a heightened risk for decompensation of their mental states. In part, such decompensation may be the result of poor adherence/compliance to a personalized patient-specific care plan. Persons with other health conditions have similar challenges in maintaining a health state and avoiding a health-deteriorated state.

What is needed is a system and method providing detection of deterioration of a person's health in other than in-patient care settings, such as in-home settings, and proactively offering assistance, guidance, coaching and/or prompting, e.g., such as established coping mechanisms for persons with mental health issues, to address a current deteriorated health state and/or to avoid a deteriorated health state, based on information in personalized care plans custom-tailored to the person. The present invention fulfills these needs, among others.

### SUMMARY

The present invention provides a system and method providing detection of deterioration of a person's health in other than in-patient care settings, such as in-home settings, and proactively offering advice, coaching, guidance and/or other prompting designed to address a deteriorated (e.g., decompensated) state, or to avoid a deteriorated state, based on a personalized care plan for the person. Accordingly, the present invention may be configured, for example, to monitor a patient's mental health and assess a current mental health state to identify deterioration/decompensation and/or a level of risk of deterioration. This is done through the use of sensors for multi-faceted data collection, and analysis of collected data relating to characteristics usable to assess the person's health, e.g., the person's biometric data (e.g., facial affect, sleep pattern, blood pressure, heart rate, heart rate variability), activity tracking data, environmental data, and interactive health assessment screening data, which may be gathered in response to delivery of an interactive health assess meeting screening via an Assistive Device in accordance with the present invention.

As part of routine tasks, an intelligent health assistant device (“Assistive Device”) provides prompting to take action to avoid the deteriorated state in accordance with the person's personalized care plan, which may be developed by a clinician, of by prior self-identification of specific actions by the person. By way of example, the Assistive Device can guide the person to attend clinician visits, to engage in wellbeing/exercise/other activities, to take medications, etc., to maintain the person's health and/or to avoid the deteriorated health state, in accordance with information in the person's personalized care plan, which may be developed by a clinician, of by prior self-identification of specific actions by the person.

In the event of detection of a deteriorated health state (e.g., a current deteriorated state, or a current heightened risk of entering a deteriorated state, collectively referred to herein as in a deteriorated state, for simplicity), the assistive device in accordance with the present invention provides prompting to take action to address the deteriorated state in accordance with the person's personalized care plan. By way of example, the Assistive Device can guide the person to engage in stabilizing coping mechanism activities, to contact and engage with an assistive resource (e.g., a person for providing support through dialog), etc. In embodiments intended to address mental health issues for mental health patients, the personalize care plan may include tasks identified by a clinician as part of the person's post-discharge safety plan.

Accordingly, the Assistive Device and system of the present invention can improve a person's adherence/compliance to the person's personalized care plan—e.g., provider visit schedules, activities, and medications, as well as provide effective support to patients in accordance with the person's personalize care plan in the event of crisis/decompensation/health deterioration. This can be especially useful in the case of persons with mental health issues, and particularly to provide readily-accessible/on-demand/immediate support to a person during the initial three months following discharge from a psychiatric facility, when mental health risks are regarded to be the highest.

In one aspect of the present invention, a special-purpose health assistive device is provided that has a form factor of a common household/workplace item, such as a mirror, lamp, glass, glasses, etc. to be used in substitution for a conventional such item. In an exemplary embodiment, the Assistive Device is configured as a mirror that displays not only an image of the person/patient, but also an image of a computer-generated avatar image with which the person may communicate/converse in a dialog session, to provide for a particularly welcoming and engaging experience for the person.

### BRIEF DESCRIPTION OF THE FIGURES

An understanding of the following description will be facilitated by reference to the attached drawings, in which:

FIG. 1 is a schematic diagram of an exemplary network communications environment in which an intelligent health assistant device may be deployed in accordance with an exemplary embodiment of the present invention;

FIG. 2 is a front view of a special-purpose intelligent health assistant device in accordance with an exemplary embodiment of the present invention;

FIG. 3 is a block diagram an intelligent health assistant device in accordance with an exemplary embodiment of the present invention;

FIG. 4 is a block diagram of exemplary health assistant management system in accordance with an exemplary embodiment of the present invention; and

FIG. 5 is a flow diagram illustrating a method for monitoring a person's health and providing in accordance with the present invention.

### DETAILED DESCRIPTION

The present invention provides a system and method providing for monitoring of a person's health in other than in-patient care settings, such as in-home settings, and proactively offering assistance, guidance, coaching and/or prompting, e.g., such as established coping mechanisms for persons with mental health issues, to address a current deteriorated health state and/or to avoid a deteriorated health state, based on information in personalized care plans custom-tailored to the person. More particularly, the present invention provides an artificial intelligence-based intelligent health assistant device and system that are more effective and cost-efficient than having a team of care providers manually supporting patients, while also allowing for regular, e.g., 24 hours/day and 7 days/week, monitoring and/or support to the person. The present invention can gather both objective data (e.g., directly from sensors worn by the patient, local to the patient, or remote from the patient) and subjective data (e.g., patient responses to questions, questionnaire/assessment tools, etc. that are captured by sensors) usable to assess a person's health state, and can do so without direct involvement by a healthcare provider, which inherently makes it more robust and reliable, less susceptible to errors and inefficiencies, and also less intrusive compared to an actual person constantly checking on a person/patient.

Further, the intelligent health assistant device is configured to provide help to a person in accordance with that person's personalized care and/or safety plan. More particularly, the device communicates prompts to the patient to take actions identified in the person's personalize care plan (e.g., safety plan), based on sensor data and/or an assessment of the person's current health state. The device may communicate prompts intended to aid a person in complying with a care plan to avoid a deteriorated state, e.g., by taking prescribed medication, attending clinician visits, engaging in certain activities intended to avoid a deteriorated state, etc. Additionally, if the device detects a deteriorated state, the device may communicate prompts intended to aid a person to address/mitigate/react to a current deteriorated state, e.g., by advising use of a particular coping mechanism, engaging in an activity or a mindfulness exercise, and using/connecting with an assistive resource, etc., in accordance with a predefined care/safety plan stored by the system.

Accordingly, an assistive device in accordance with the present invention can function somewhat like a clinician and/or as a personal emotional coach, by suggesting coping mechanisms, mindfulness exercises, and even connecting users to relevant resources when needed. The system may provide insights into emotional patterns and triggers.

Further, an assistive device in accordance with the present invention can function as a health monitoring device. By analyzing biometrics and emotional state, the device can identify potential health risks and suggests preventative measures. It can detect progress towards health goals, motivating users to make positive lifestyle changes.

Further, an assistive device in accordance with the present invention can function as a personalized information hub. The device can use/display/provide weather forecast information, reminders for routine tasks consistent with a care plan, and/or information tailored to the user's emotional state, such as daily affirmations, recommendations, etc., based on sensor data and the care plan. If the device detects that a user appears stressed/deteriorated, for example, calming affirmations and/or mindfulness prompts may be provided. If the system detects that a user is happy/not stressed/deteriorated, weather updates for an outdoor activity may be provided, as a general preventative measures, if the care plan indicates that such an outdoor activity is part of the person's care plan.

Accordingly, the device and system of the present invention empower the user to understand emotions, manage stress, and make informed decisions about health and happiness. Further, the device and system offer personalized support to the user based on an emotional or other health state, in accordance with the person's personalize care plan. By way of example, the device/system in accordance with the present invention can be used for mental health monitoring, therapy and coaching, for personal use for self-awareness and emotional wellbeing, for stress management in workplaces, and/or for personalized healthcare and early intervention, for a wide variety of health conditions.

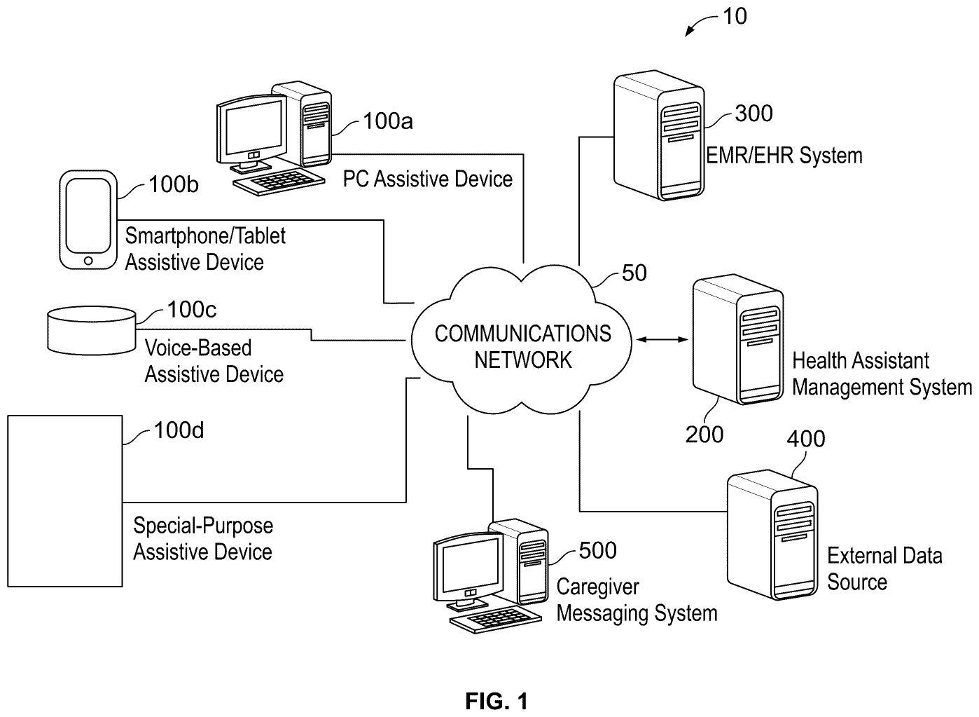

According to illustrative embodiment(s) of the present invention, various views are illustrated in FIGS. 1-5 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

### System Environment

An exemplary embodiment of the present invention is discussed below for illustrative purposes. FIG. 1 is a schematic diagram of an exemplary network communications environment 10 in which an intelligent health assistant device (“Assistive Device”) may be deployed in accordance with an exemplary embodiment of the present invention. As shown in FIG. 1, the exemplary network environment 10 includes conventional computing hardware and software for communicating via a communications network 50, such as the Internet, etc., using Assistive Devices 100a, 100b, 100c, 100d (collectively, 100) in accordance with the present invention, which may be, for example, one or more personal computers/PCs, laptop computers, tablet computers, smartphones, voice-based digital assistant computing devices capable of receiving inputs from humans in the form of spoken words/speech or other computing device hardware including computerized/networked communication hardware/software/functionality, such as computer-based kiosks, etc. Examples of voice-based digital assistant computing devices include Amazon Alexa-based devices, such as the Echo and Dot devices manufactured and/or sold by Amazon Technologies, Inc., the Google Home device manufactured and/or sold by Alphabet, Inc., and the Sonos One devices manufactured and/or sold by Sonos, Inc.

In accordance with a certain aspect of the present invention, one or more of the Assistive Devices 100a, 100b, 100c, 100d may store and execute an “app” or other purpose-specific software in accordance with the present invention, although this is not required in all embodiments.

In accordance with the present invention, the network computing environment 10 further includes a Health Assistant Management System 200 in accordance with the present invention, which may be configured as a web server in a client/server environment, or as another cloud-based device capable of exchanging data and/or performing functions of an Assistive Device, and/or in collaboration with at least one Assistive Device, in accordance with the present invention. In certain embodiments, some or all of the functionality of an Assistive Device 100 may be provided by working in concert with the Health Assistant Management System 200 or other components within the network computing environment 10.

In accordance with the present invention, the exemplary network computing environment 10 further includes an Electronic Medical Record/Electronic Health Record (EMR/EHR) System 300. The EMR/EHR System 300 is operatively connected to the Assistive Devices 100 and/or Health Assistant Management System 200 via the communications network 50, so that a person's clinician-developed care plan and/or safety plan stored in a patient record of the EMR/EHR System 300 can be shared with an Assistive Device 100 and/or Health Assistant Management System 200 for the purposes described herein. Optionally, information gathered from/via an Assistive Device 100 and/or Health Assistant Management System 200 may also be shared with the EMR/EHR System 300 so relevant health/incident-related information can be incorporated into the corresponding patient's medical record/chart in the EMR/EHR System 300. Such EMR/EHR systems are commercially available in the marketplace, and are beyond the scope of the present invention, and thus are not discussed in greater detail herein. By way of example, the Assistive Device 100 and/or Health Assistant Management System 200 may be configured with, or to interface with, software for automating data integration with a Cerner, Epic, AllScripts or other EMR/EHR System 300. These systems may be existing or otherwise generally conventional systems, at least in part, including conventional software and web server or other hardware and software for communicating via the communications network 50.

In accordance with the present invention, the network computing environment 10 further includes an External Data Source 400. The External Data Source 400 may be any independent source of data/sensor data that contains data useful in the context of the present invention. The External Data Source 400 is operatively connected to the Assistive Devices 100 and/or Health Assistant Management System 200 via the communications network 50, so that data from the External Data Source 400 may be used by the device and system in accordance with the present invention, e.g., to be included in an analysis of a person's current health state, or to be used to address a person's current deteriorated health state, and/or to be used to recommend an activity/task/exercise, etc. in accordance with a person's care plan, e.g., to avoid a deteriorated health state. The External Data Source 400 may have any suitable hardware configuration, such as a web server in a client/server environment, or another cloud-based device, database or other data store, or as an external device such as a fitness tracker device or other wearable device, an internet-of-things device, etc. The External Data Source 400 may be any source/repository of any relevant data, such as biometric data, wearable/fitness tracker data, activity data, GPS/movement/location data, weather forecast data, environmental data (humidity, temperature, etc.), etc. These External Data Source 400 may be existing or otherwise generally conventional systems, at least in part, including conventional hardware and software and web server or other hardware and software for communicating via the communications network 50. Such data sources/devices are well known in the art and beyond the scope of the present invention, and thus are not discussed in detail herein.

In accordance with the present invention, the network computing environment 10 further includes a Caregiver Messaging System 500. The Caregiver Messaging System 500 may be any communications device, such as a personal computer/PC, tablet computer, smartphone, or telephone, that allows a caregiver to receive a data or other communication (e.g., e-mail, text message, telephone call, etc.) in relation to the person/patient. By way of example, the caregiver may be a clinician, a home health care nurse, or a layperson, such as someone identified in a clinician's safety plan, or someone self-identified by the person/patient as a person that should be contacted to serve as an assistive resource to the person in the event of the person's deteriorated health state (or to avoid a deteriorated health state). The Caregiver Messaging System 500 is operatively connected to the Assistive Devices 100 and/or Health Assistant Management System 200 via the communications network 50, so that a transmitted message may be received in appropriate circumstances. A Caregiver Messaging System 500 may be existing or otherwise generally conventional systems, at least in part, including conventional hardware and software for communicating via the communications network 50. Such devices are well known in the art and beyond the scope of the present invention, and thus are not discussed in detail herein.

It should be noted that in FIG. 1, the Assistive Devices 100a, 100b, 100c, 100d and the Health Assistant Management System 200 are shown as separate and discrete systems for illustrative clarity, but that in other embodiments, the functionality of these independent components (and associated hardware and/or software) may be integrated in whole or in part into an Assistive Device and/or the Health Assistant Management System 200. In other words, various embodiments of the present invention may involve an Assistive Device 100 without a Health Assistant Management System 200, a Health Assistant Management System 200 without an Assistive Device 100, or both an Assistive Device 100 and a Health Assistant Management System 200 that work in concert to provide the functionality contemplated herein. In certain embodiments, all functionality of the Assistive Device 100 may be integrated into the Health Assistant Management System 200, without a need to communicate via the communications network 50, such that all tasks are performed at the Health Assistant Management System 200, and vice versa. Accordingly, it should be appreciated that the description with respect to FIG. 1 and/or the exemplary embodiment is for illustrative purposes only, and not limiting.

### Assistive Device

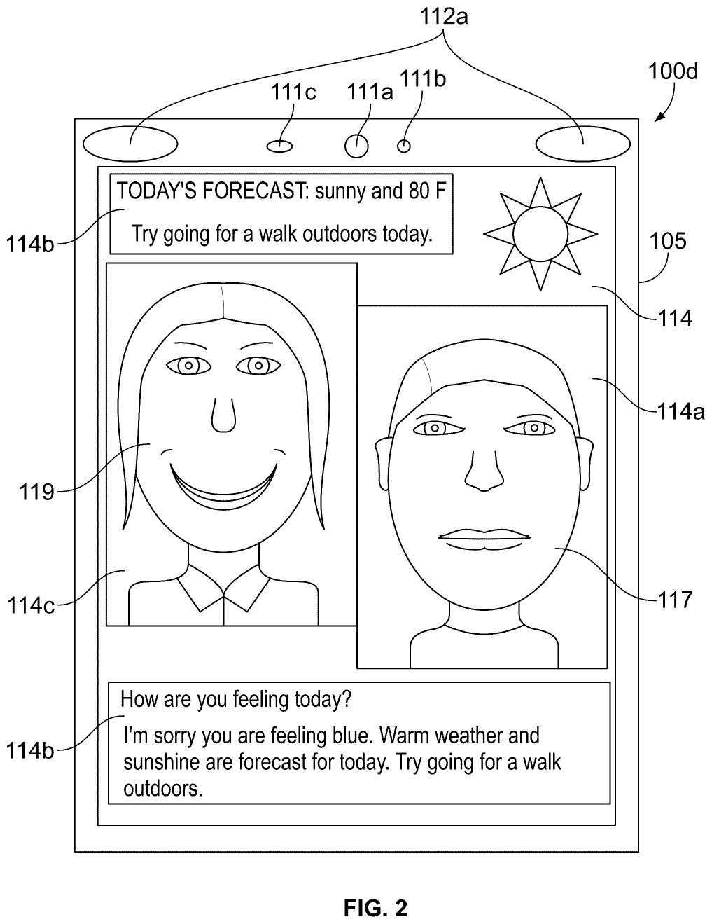

FIG. 2 is a front view of a special-purpose intelligent health assistive device 100d in accordance with an exemplary embodiment of the present invention. In accordance with the present invention, the special-purpose intelligent health assistant device is configured as a common/everyday-type item common in a person's household/work/other environment that serves a primary purpose according to its nature, and a secondary purpose in accordance with the present invention. For example, the special-purpose intelligent health assistant 100d may be configured as a household mirror, and function as a mirror to display an image, but also function in accordance with the present invention. By way of further example, in this exemplary embodiment, the special-purpose intelligent health assistant 100d may be configured as other common household/environmental-type objects commonly found in hospital room, home and/or work environments, such as a mirror, lamp, a glass surface (e.g., of an appliance, door, window, etc.) a pair of glasses, a fitness tracker/wearable device, that function like an ordinary such objects, but also include additional components and functionality to function in accordance with the present invention. In accordance with the present invention, these devices are provided in the patient's environment, so that they can be used to monitor the patient. Accordingly, monitored persons can interact with this system as part of their daily routines, often passively, using wearable devices including fitness trackers (that focus on physical data and functionality) and everyday household/personal items including embedded sensor and/or computational devices utilizing machine vision and/or voice user interfaces in accordance with the present invention.

The exemplary special-purpose intelligent health assistant 100d of FIG. 2 is configured as a common household object, namely, mirror-type furniture/houseware, and thus could be placed and used in a person's home, workplace or other environment in lieu of a conventional mirror. Accordingly, the Assistive Device 100d of FIG. 2 is described below for illustrative purposes in the context of a mirror, but it should be noted that in other embodiments, some of the described components may be omitted. For example, when configured as a lamp, the assistive device 100 may exclude a camera and display and the associated functionality, but may otherwise function in accordance with the description provided below, as will be appreciated by those skilled in the art.

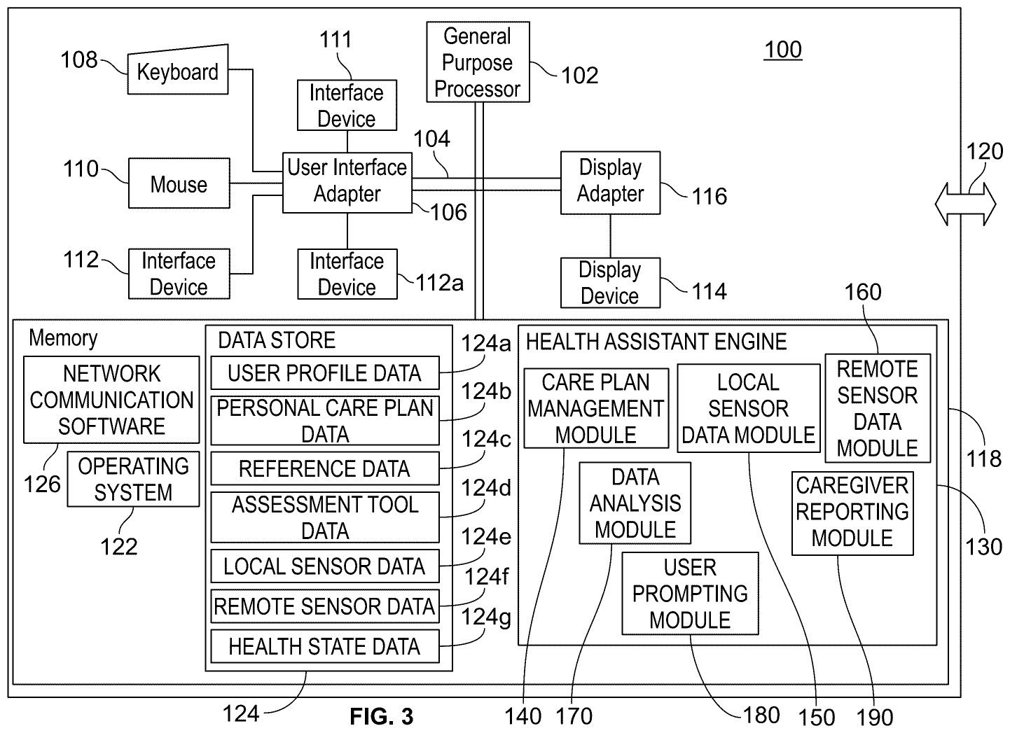

Further, it should be noted that the other exemplary Assistive Devices 100a, 100b, 100c shown in FIG. 1 are special-purpose devices configured in accordance with the present invention, but may include conventional hardware and software of typical conventional communication devices, such as a personal computer, tablet computer, smartphone, voice-based assistant device, etc. These devices may include the hardware, software and functionality described below in relation to the exemplary Assistive Device 100d of FIG. 2. FIG. 3 is a block diagram an Assistive Device 100 in accordance with an exemplary embodiment of the present invention, and is illustrative of all such assistive devices 100a, 100b, 100c, 100d, although in certain embodiments of the present invention some components may nevertheless be omitted, as noted above.

Referring now to FIG. 2, an exemplary special-purpose intelligent health assistant device 100 (Assistive Device 100d) is shown. The device is representative of all intelligent health assistive devices (100a, 100b, 100c, 100d, collectively, 100) in that it is a computerized health assistive device for monitoring a person's health and providing care plan-based guidance to the person. In accordance with the present invention, the Assistive Device 100 comprises a housing 105 that houses a processor and a memory operatively connected to the processor, as described in greater detail below with reference to FIG. 3.

The Assistive Device 100d further includes at least one sensor 111 supported on the housing 105. The at least one sensor 111 is configured to gather data relevant to assessment of the person's health. In this exemplary embodiment, the at least one sensor includes a camera 111a and a microphone 111b. The camera 111a is adapted to capture still and/or videographic images of the person, and the microphone is adapted to capture voice samples and/or voice responses from the person, and other environmental sound input from the environment of the person. Accordingly, these sensors are local to the device 100, i.e., at and/or integrated with the Assistive Device 100. The Assistive Device 100 may include one or more other sensors 111c adapted to capture data relative to at least one characteristic usable to assess a person's health state (which may include a current health state or an expected health state in the near term). For example, such sensors may include a remote photoplethysmography (RPPG) sensor, a blood pressure sensor, a BMI sensor, body composition sensor, body temperature sensor, a heart rate sensor, a heart rate variability sensor, a biometric sensor, a weather sensor, an environmental sensor, a wearable device sensor, and an internet-of-things device sensor. Any suitable sensor for capturing data relative to any characteristic usable to assess a person's health state may be used in accordance with the present invention. Such sensors are well known in the art and beyond the scope of the present invention, and thus are not discussed in detail herein.

Notably, the Assistive Device/system including an Assistive Device 100 or HAMS 200, may also be configured to receive data from external devices and/or data sources, e.g., those associated with remote sensors that are not local to the device 100, i.e., those that are in/at remote locations away from the Assistive Device 100, and that are not integrated with/supported on the housing of the Assistive Device 100. Such sensors are adapted to capture data relative to at least one characteristic usable to assess a person's health state (which may include a current health state or an expected health state in the near term). For example, such sensors may include a remote photoplethysmography (RPPG) sensor, a blood pressure sensor, a heart rate sensor, a BMI sensor, a heart rate variability sensor, a biometric sensor, GPS/movement/location sensor, a weather (e.g., temperature, humidity) sensor, an environmental sensor, a wearable device sensor, and an internet-of-things device sensor. Such sensors are well known in the art and beyond the scope of the present invention, and thus are not discussed in detail herein. By way of example, some or all of these sensors may be associated with a fitness tracker or other wearable device or a smartphone of the person. By way of further example, some or all of these sensors may produce data that are stored at an External Data Source 400 that is accessible to the Assistive Device/system of the present invention for the purposes described herein.

The Assistive Device 100 further includes a user interface device 112 supported on the housing 105. The user interface device 112 is operatively connected to the processor and operable to provide at least one of an audible prompt and a visual prompt to the person. In this exemplary embodiment, the user interface device comprises a display device 114 (operable to provide a visual prompt) and a pair of speakers 112a (operable to provide an audible prompt) supported on the housing 105. Optionally, the display device 114 may be a touchscreen display device, such that it is adapted to receive user input from the person as touch input on the touchscreen display 114. For example, the speakers 112a may be used to provide an audible prompt that may be a question intended to elicit a response (e.g., “How are you feeling today”), e.g., as part of a health assessment tool, such as a SIGECAPSD (sleep, interest, guilt, energy, concentration, appetite, psychomotor, suicide, depression), or common evidence-based screening tools such as the PHQ (patient health questionnaire). Alternatively, the audible prompt may be spoken (or be computer-simulated spoken) words intended to instruct or encourage the person to take an action, such as to perform a particular coping mechanism, engage in a particular activity, perform a mindfulness exercise, and/or access/contact an assistive resource (e.g., such as to call a support person to have a discussion), in accordance with the person's care plan, as discussed in greater detail below.

Because this Assistive Device 100d is configured as a mirror, the display device 114 defines a first display area 114a configured to display an image of the person 117 captured by the camera 111a, so that a person using the mirror can view his/her own image, akin to a reflection, to provide a mirror-like user experience.

In this exemplary Assistive Device embodiment, the display device 114 further defines a second display area 114b configured to display a visual prompt to the person, e.g., to perform a certain action, as described below. For example, the visual prompt may be a question intended to elicit a response (e.g., “How are you feeling today?”), e.g., as part of a health assessment tool. Alternatively, the displayed visual prompt may be text intended to instruct or encourage the person to take an action, such as perform a particular coping mechanism, engage in a particular activity, perform a mindfulness exercise, and/or access/contact an assistive resource (e.g., such as to call a support person to have a discussion), in accordance with the person's care plan, as discussed in greater detail below.

In this exemplary embodiment, the display device 114 further defines a third display area 114c configured to display an image of an avatar 119, e.g., as animated to appear to speak the audible and/or visual prompts. For example, the use of a computer-generated avatar for this purpose can provide the person with a friendly and helpful dialog-type experience, which can mimic a conversational experience with a clinician, caregiver, friend, or coach, and be useful in encouraging the person to engage and communicate with the Assistive Device/system for the purposes described herein. The avatar may be an image of an AI-based/simulated assistant (e.g., a digital human representation). Alternatively, or additionally, the display device may include a surface that allows for haptic feedback.

In this embodiment, the housing 105 further houses a data analysis module operable to analyze data from at least one sensor and to determine as a function of the data whether a (current) health state of the person is indicative of a deteriorated health state (which includes a heightened risk or entering a deteriorated health state even if not in a current deteriorated health state), and a user prompting module operable to identify a specific action to be taken by the person, by referencing a care plan stored in a memory of the device, and to communicate the specific action to be taken via the user interface device as a prompt to the person to address or avoid the deteriorated health state. Again, it is noted that in other embodiments, some or all of these components and associated functions may be performed at a remotely located Health Assistant Management System 200, or by acting in concert therewith.

FIG. 3 is a block diagram showing an exemplary Health Assistive Device (HAD) 100 in accordance with an exemplary embodiment of the present invention. The HAD 100 is a special-purpose computer system that includes not only conventional computing hardware storing and executing conventional software enabling operation of a general-purpose computing system, such as operating system software 122, network communications software 126, but also specially-configured computer software for carrying out at least one method in accordance with the present invention. By way of example, the network communication software 126 may include conventional web server software, and the operating system software 122 may include iOS, Android, Windows, Linux software.

Accordingly, the exemplary HAD 100 of FIG. 3 includes a general-purpose processor, such as a microprocessor (CPU) 102 and a bus 104 employed to connect and enable communication between the processor 102 and the components of the presentation system in accordance with known techniques. The exemplary HAD 100 includes a user interface adapter 106, which connects the processor 102 via the bus 104 to one or more interface devices, such as a keyboard 108, mouse 110, a camera 111a (particularly a user-facing camera), microphone 111b, speakers 112a and/or other interface devices such as a touch sensitive screen or pad, etc., as well as to one or more sensors used to capture data relative to at least one characteristic usable to assess a person's (current or future) health state, such as a remote photoplethysmography (RPPG) sensor, a blood pressure sensor, a heart rate sensor, a heart rate variability sensor, a biometric sensor, a weather sensor, an environmental sensor, a wearable device sensor, and an internet-of-things device sensor. The bus 104 also connects a display device 114, such as an LCD screen or monitor, to the processor 102 via a display adapter 116. The bus 104 also connects the processor 102 to memory 118, which can include a hard drive, RAM or other solid-state memory, diskette drive, tape drive, etc.

The HAD 100 may communicate with other computers or networks of computers, for example via a communications channel, network card or modem 120. The HAD 100 may be associated with such other computers in a local area network (LAN) or a wide area network (WAN), and may operate as a server in a client/server arrangement with another computer, etc. Such configurations, as well as the appropriate communications hardware and software, are known in the art.

The HAD 100 is specially-configured in accordance with the present invention. Accordingly, as shown in FIG. 3, the HAD 100 includes computer-readable, processor-executable instructions stored in the memory 118 for carrying out the methods described herein. Further, the memory 118 stores certain data, e.g., in one or more databases or other data stores 124 shown logically in FIG. 3 for illustrative purposes, without regard to any particular embodiment in one or more hardware or software components.

Further, as will be noted from FIG. 3, the HAD 100 includes, in accordance with the present invention, a Health Assistant Engine (HAE) 130, shown schematically as stored in the memory 118, which includes a number of additional modules (e.g., components) providing functionality in accordance with the present invention, as discussed in greater detail below. These modules may be implemented primarily by specially-configured software including microprocessor-executable instructions stored in the memory 118 of the HAD 100. Optionally, other software may be stored in the memory 118 and and/or other data may be stored in the data store 124 or memory 118.

As shown in FIG. 3, the exemplary embodiment of the HAD 100/HAE 130 includes a Care Plan Management Module (CPMM) 140. The CPMM 140 is responsible for obtaining and/or storing suitable care plan data 124b in association with a particular person identified by that person's user profile data 124a. In certain embodiments, the CPMM 140 may be responsible for creating a user profile for each person and storing it in the user profile data 124a. By way of example, the user profile data may identify a person's name, age, weight, gender, insurance information, demographic information, hobbies/interests, etc., and the personal care plan data 124b may identify clinician-identified actions to be taken by the person, e.g., in the event of a deteriorated state, or to avoid a deteriorated state, or other relevant information. For example, those actions and information may be taken from a clinician-developed safety plan before discharge of a person treated for mental health or behavior health issues, or otherwise be part of a person's clinician-developed care plan. For example, actions may be one or more coping mechanisms, activities, or mindfulness exercises to be engaged in, or assistive resources (e.g., people or other resources) to be contacted/accessed that can aid in addressing/mitigating/avoiding a deteriorated state.

The personal care plan data 124b may also include information relating to other aspects of a person's care plan, such as data identifying treating clinicians, clinician visit/appointment schedules/dates, medications/medication schedules, etc., that assist a person in avoiding a deteriorated state. Additionally, the personal care plan data 124b may include information self-identified by the person as actions to be taken in the event of a deteriorated state, or to avoid a deteriorated state.

In certain embodiments, the CPMM 140 is responsible for communicating data with an external EMR/EHR System 300 to retrieve care plan data for storage, e.g., from a person's medical record, and/or to work with other components of the HAE 130 to provide audible or visible prompts to the user and to gather user responses, e.g., as to the person's self-identification of preferred coping mechanisms, activities, or mindfulness exercises to be engaged in, or assistive resources (e.g., people or other resources) to be contacted/accessed that can aid in addressing/mitigating/avoiding a deteriorated state. This is performed according to logic/questions incorporated into the CPMM 140.

In accordance with the present invention, the exemplary embodiment of the HAE 130 shown in FIG. 3 also includes a Local Sensor Data Acquisition Module (LSDAM) 150. The LSDAM 150 is responsible for causing, or otherwise receiving, data captured by a local sensor of the device 100 in relation to at least one characteristic usable to assess the person's health state. For example, the LSDAM 150 may cause a camera device to capture an image of a current user/operator of the HAD 100, or to cause a microphone device to capture a voice sample, spoken responses, or sounds in the environment of the user, or may cause an RPPG sensor to capture psychophysiological data including data relating to heart rate variability, respiration rate, blood pressure and oxygenation, quality of sleep, heart rhythm disturbances, and also mental stress and drowsiness. Additionally, for example, the LSDAM 150 is responsible for causing the camera device to capture an image of the user/operator of the HAD 100 for display via the display device in a mirror-type special-purpose assistive device, or for gathering of data for analysis of the person's health state. The LSDAM 150 is further responsible for storing associated data captured by sensors local to the HAD 100 as Local Sensor Data 124e in the data store 124 of the HAD 100.

In accordance with the present invention, the exemplary embodiment of the HAD 100/HAE 130 shown in FIG. 3 also includes a Remote Sensor Data Acquisition Module (RSDAM) 160. The RSDAM 160 is responsible for causing, or otherwise receiving, data captured by a remote sensor that is not physically integrated into the device 100, but that is capable of gathering data in relation to at least one characteristic usable to assess the person's health state. For example, the RSDAM 160 may cause a remote camera or microphone device to capture image or voice data, or a remote sensor to capture biometric, environmental, weather or other data, e.g., from a wearable device directly, or from an External Data Store 400 storing data at a location accessible via a communications network. Additionally, for example, the RSDAM 160 is responsible for storing associated data captured by sensors remote from the HAD 100 as Remote Sensor Data 124f in the data store 124 of the HAD 100.

Collectively, the LSDAM 150 and the RSDAM 160 are responsible for gathering data relative to at least one characteristic usable to assess a person's health state. In accordance with the present invention, the exemplary embodiment of the HAD 100/HAE 130 shown in FIG. 3 further includes a Data Analysis Module (DAM) 170 that is operable to analyze such data to assess a current health state of the person as a function of the captured data. In certain embodiments, the DAM 170 is configured to analyze recently captured data to assess a person's health state, and determine whether the person is presently in a deteriorated health state (which includes a heightened risk of entering a deteriorated health state). Any available data usable to assess a person's health state may be used in this analysis. Commercially-available services and technologies exist for analyzing data, including sensor data relating to biometric data, activity/movement/location data, environmental data, and substantive responses in statements and/or responses to questions of assessment tools, and determining whether the person is in a deteriorated health state and/or at risk of a deteriorated health state, and any known hardware, software and/or technique may be used for this purpose. Accordingly, such analyses are beyond the scope of the present invention and are not discussed in detail herein.

In certain embodiments, recently captured data may be compared to Reference Data 124c, which may be stored in the memory 124, as part of the analysis. Reference Data 124c may include generic data that is not specific to any particular person, or previously-captured data specific to the particular person, e.g., baseline data for a pre-discharge or other known health state, to perform the analysis. By way of example, one or more Assistive Devices may be employed as a wearable device on a user, or as a device in the user's environment (e.g., as part of a mirror in a hospital room) during an in-patient stay in a healthcare facility. Personalized user-specific data may thereby be captured and associated with the user, e.g., when the user is in a poor mental state and/or after the user has been treated and is in a healthy mental state, to gather baseline/benchmark data for subsequent use after patient discharge as previously-captured data for subsequent data analysis purposes.

In certain embodiments, spoken words may be captured and processed with natural language processing to determine the content of the spoken words, and such content/spoken words may be considered as part of the analysis to assess the person's health state and determine whether the person is presently in a deteriorated health state.

In certain embodiments, the DAM 170 may cause data to be transmitted via the communications network 50, so that all or a portion of the analysis of the data is performed outside of the DAM 170, e.g., at the Health Assistant Management System 200, or otherwise, e.g., using commercially-available systems or services. By way of example, sensor data indicating difficulties in sleeping (e.g., as indicated my movement/GPS/location data), a lack of interests, a high level of guilt (e.g., derived from tone of voice or natural language processing of vocal responses), a lack of concentration, a loss of appetite, a lack of motivation/listlessness (e.g., derived from tone of voice or natural language processing of vocal responses), thoughts of suicide (e.g., as indicated in questions of an assessment tool), a depressive state, etc. may be indicative of a deteriorated health state. More particularly, the present invention provides that any combination of data available and useful in assessing the person's health state, and particularly whether the person is presently in a deteriorated health state, or at heightened risk of entering a deteriorated health state, may be used in accordance with the present invention. Any suitable sensor data and technologies, and any suitable analysis methodologies may be used in accordance with the present invention. Various sensors, technologies, and analysis methodologies for assessing the person's health state, and particularly whether the person is presently in a deteriorated health state, or at heightened risk of entering a deteriorated health state are known in the art, and thus are not discussed in greater detail herein.

The results of the DAM's analysis of the current health state of the user (e.g., whether or not the person is in a deteriorated state) is stored in the data store 124 as Health State Data 124g.

In accordance with the present invention, the exemplary embodiment of the HAD 100/HAE 130 shown in FIG. 3 further includes a User Prompting Module (UPM) 180 that is operable to identify a specific action to be taken in/in view of the Personal Care Plan Data 124b stored in the memory 118, and to communicate the specific action to be taken via a user interface device as a prompt to the person, e.g., by display of a visual prompt via a display screen, or providing an audible prompt via speakers, etc. The UPM 180 may act in concert with the DAM 170 and/or the LSDAM 150 and/or RSDAM 160 to identify/determine which prompts are appropriate at a given point in time, e.g., as a function of the sensor data and/or data analysis by the DAM 170 and associated Health State Data 124g. Notably, predefined logic and/or generative artificial intelligence (AI) technologies may be used to generate suitable questions/guidance/prompts for presentation to the person, based on the person's responses to prompts and data gathered and/or analyzed in relation to an assessment of the person's health. Predefined logic and/or generative AI techniques may also be used to determine which of various actions in a care plan are appropriate for presentation to the person at any given time, e.g., based on the person's responses to prompts and data gathered and/or analyzed in relation to an assessment of the person's health. For example, sensor data and/or the DAM's determination may result in the UPM 180 prompting the person to perform routine tasks designed to avoid a deteriorated state, such as providing reminders for clinician visits, to take medications, to provide daily affirmations, inspirational recommendations, advice or messages, or to perform certain tasks (e.g., taking a walk outdoors) designed to avoid a deteriorated state (and/or promote personal wellbeing), according to actions identified in the person's personal care plan (stored as Personal Care Plan Data 124b), which may be performed when the person is not in a deteriorated state, or regardless of whether the person is in a deteriorated state. By way of further example, the DAM's determination that a person is in a deteriorated state may result in the UPM 180 prompting the person to perform actions designed to address/mitigate a deteriorated state, such as performing certain coping mechanism exercises or mindfulness exercises, engaging in certain activities, or contacting/accessing certain assistive resources (e.g., people or other resources) to be that can aid in addressing/mitigating/avoiding a deteriorated state.

In certain embodiments, the HAD 100 may store assessment tool information such as psychological assessment tool, health inventory, or other questionnaires, as Assessment Tool Data 124d in the data store 124, and the UPM 180 may prompt the user to provide responses to questions according to such assessment tool data. Various assessment tools and health inventory and other questionnaires for assessing a person's health state are known in the art, and beyond the scope of the present invention, and thus are not discussed in detail herein. In such embodiments, the person's responses are captured by sensors (e.g., local sensors), captured by a Sensor Data Module, 150, 160, and stored as sensor data 124e, 124f, and may be accessed and considered as part of the analysis of health state performed by the DAM 170.

In accordance with the present invention, the exemplary embodiment of the HAD 100 shown in FIG. 3 also includes a Caregiver Reporting Module (CRM) 190. The CRM 190 is responsible for notifying a caregiver (which may be a clinician, layperson or other person identified as an assistive resource contact person in the care plan data). This may involve the use of any suitable logic. For example, a determination of the DAM that the user is in a deteriorated state, or at risk for entering a deteriorated state due to certain present conditions, may result in not only prompting to the person to contact a certain person identified as an assistive resource that can provide moral support/coaching/dialog, etc. in the event of a deteriorated state or risk of a deteriorated state, but also in sending of a message to the caregiver/assistive resource so that the caregiver is notified of the person's state and encourage to provide support to the person in the person's time of need for support. This may involve sending data communications via the communications network 50 to the Caregiver Messaging System 500, e.g., as a telephone call, e-mail message, text message, etc.

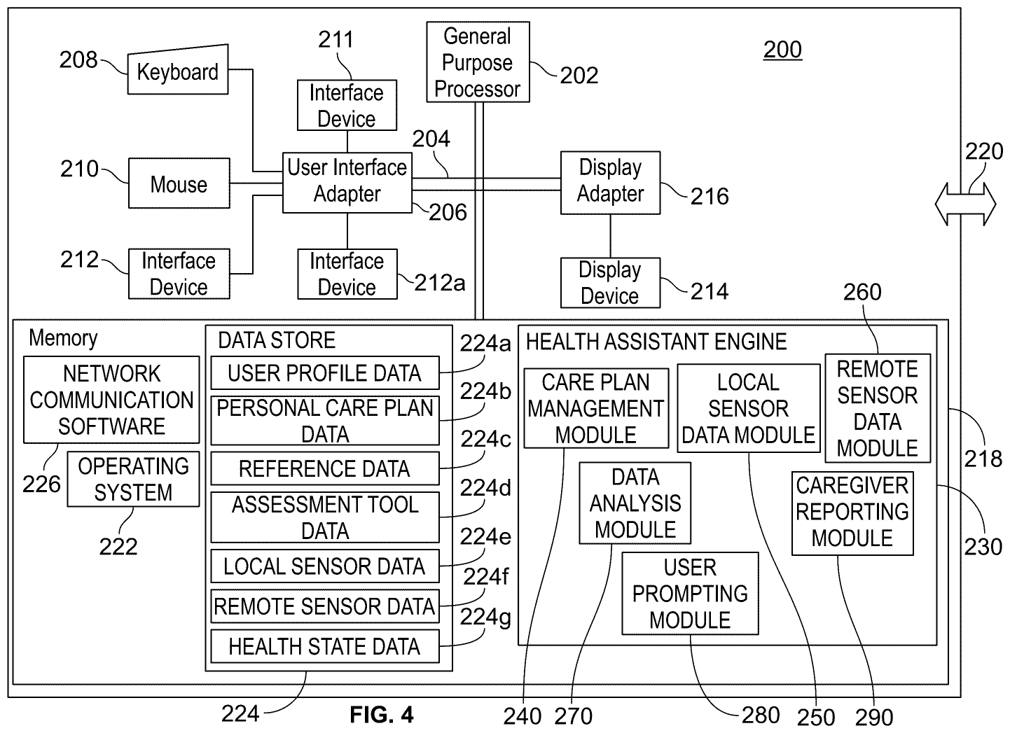

FIG. 4 is a block diagram of exemplary Health Assistant Management System (HAMS) 200 in accordance with an exemplary embodiment of the present invention. As noted above, the HAMS 200 may be similar in structure to the Assistive Device 100, and therefor may include all or fewer than all of the components of the Assistive Device 100, which may function in the same or a similar manner as the Assistive Device, as described above. Accordingly, the device/system functionality described herein may be performed entirely by the Assistive Device 100, entirely by the HAMS 200, or by the Assistive Device 100 working in concert with the HAMS 200. For example, it may be advantages to store data at the HAMS 200 and/or to perform intensive data processing tasks at the HAMS, rather than at the Assistive Device 100, as will be appreciated by those skilled in the art. Accordingly, it will be appreciated that not all of the components of the Assistive Device 100 and HAMS 200 are required in all embodiments, and that certain embodiments of the system may omit certain components from one or the other of the Assistive Device 100 and HAMS 200. Accordingly, the HAMS 200 may alternatively be described as an assistive device of health assistive device, or the assistive device 100 and HAMS 200 in combination may be considered the assistive device.

### Device/System Operation

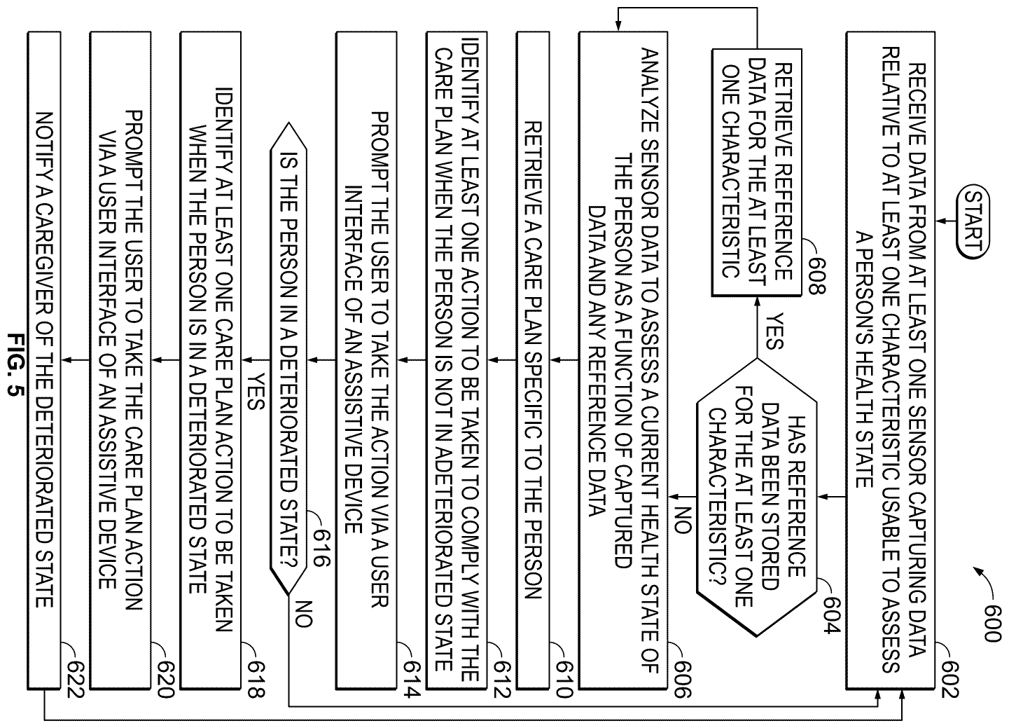

Exemplary operation of the Assistive Device/system of FIGS. 1-4 is illustrated in the flow diagram of FIG. 5, which may be implemented by actions taken by an Assistive Device 100, a Health Assistant Management System 200, or the Assistive Device 100 and HAMS 200 working in concert, that stores at least User Profile Data 124a and Personal Care Plan Data 124b, and has a Health Assistant Engine 130 including one or more modules described above or otherwise operates in accordance with the present invention. It should be noted that the method shown in FIG. 5 may be repeated periodically for a single person, and further may be performed and repeated for many different persons concurrently. As described above the User Profile Data 124a and Personal Care Plan Data 124b may be received/retrieved via data communication via the communications network 50 with an external EMR/EHR System 300, or may otherwise be gathered from a person, e.g., via questions presented to the Person via the Assistive Device 100, by the Care Plan Management Module 140, and responses captured by user interface device(s) of the Assistive Device 100.

Referring now to the exemplary method shown in the flow diagram 600 of FIG. 5, an exemplary method for monitoring a person's health and providing care plan-based guidance to the person begins with receiving data from at least one sensor capturing data relative to at least one characteristic usable to assess a person's health state, as shown at 602. As described above, sensor data may be received via the Local Sensor Data Module 150 and/or Remote Sensor Data Module, which may include receiving relevant data from an External Data Source 400 via the communications network 50. It should be noted that this may include the User Prompting Module 180 prompting the person to respond to assessment tool questions or health assessment questionnaire questions to gather additional data (via sensors) from the person that is usable to assess the person's health state.

By way of example, characteristics may include blood pressure, body mass index, heart rate, heart rate variability, breathing/respiration rate, facial affect, facial skin appearance, sleep patterns, blood oxygen saturation, body temperature, body composition, movement (e.g., accelerometer or GPS data) and/or movement patterns, outdoor temperature, weather forecast, visual aspects of a person's environment, auditory aspects of a person's environment, voice tone, voice inflection, spoken words, conversation understanding and any other biometric, behavioral, environmental or other parameter that may be usable to assess a person's health state.

Next, the method involves determining whether Reference Data 124c has been stored for the at least one characteristic, as shown at 604. If so, then the relevant Reference Data 124c for the person is retrieved from the data store 124, as shown at 608. If there is no relevant Reference Data, then no Reference Data 124c is received.

In either case, method flow continues 606, which involves analyzing the received sensor data, and any Reference Data 124c, if applicable, to assess a current health state of the person as a function of the captured sensor data (Local Sensor Data 124e and Remote Sensor Data 124f) and any Reference Data 124c. This is performed by the Data Analysis Module 170, and results in a determination of whether the person is in a deteriorated state or is not in a deteriorated state, based on the sensor data and analysis of the sensor data. The deteriorated state may be determined to be mild or more severe, and suitable actions may be determined accordingly. The results of the analysis by the DAM 170 is stored as Health State Data 124g for the person.

Next, the UPM 180 retrieves a care plan specific to the person, e.g., from the Personal Care Plan Data 124b, as shown at 610. As shown at 612, the UPM 180 identifies at least one care plan action to be taken, e.g., even when the person is not in a deteriorated state, or when the person is in a deteriorated state, as shown at 612. By way of example, this may involve providing prompts related to routine matters or tasks designed to avoid a deteriorated state, such as reminders for clinician visits, or to take medications, providing of positive affirmations or reinforcement messages, or providing of recommendations to perform certain actions, such as to perform preventative/coping mechanisms, engage in meditation exercises, certain physical activities, etc. Notably, the action may be a clinician-identified action from a clinician-developed care plan, such as a post-discharge safety plan for a person treated for mental health conditions, or the action may be an action self-identified by the person as an action to be taken in such a circumstance, e.g., to adhere to the care plan, promote wellbeing and otherwise avoid a deteriorated health state, as described above. This may involve the UPM 180 determining which of several actions of the care plan is appropriate at this time, e.g., based on the sensor data and analysis thereof.

In this exemplary method, the person is then prompted to take/perform the action via a user interface device of the Assistive Device 100, as shown at 614. This is performed by the UPM 180, and may involve the UPM 180 displaying a visual prompt message via a display device 114 of the Assistive Device 100, or providing an audible prompt message via speakers 112a of the Assistive Device 100. Accordingly, the Assistive Device 100 provides sensor data-based guidance/assistance to the person to avoid a health deteriorated state, even with the person is not currently in a deteriorated state. Notably, the action to be taken is determined consistent with the personalized care plan, and may be determined as a function of sensor data. For example, in certain circumstances, sensors providing weather condition data and/or current environmental condition data may be used to cause prompting to engage in an activity identified as a soothing activity in a person's care plan, such as a walk outdoors when sensor data indicates that the weather is warm, dry, and/or sunny.

In this exemplary method, if the person has not been determined to be in a deteriorated state, then method flow returns to 602 to allow for continued sensor data collection and analysis and prompting of the person to avoid a deteriorated state, etc., as shown at 616 and 602.

If, however, it is determined that the person is in a deteriorated health state, as then the UPM 180 identifies at least one care plan action to be taken when the person is in a deteriorated state, as shown at 616 and 618. Notably, the action may be a clinician-identified action from a clinician-developed care plan, such as a post-discharge safety plan for a person treated for mental health conditions, or the action may be an action self-identified by the person as an action to be taken in such a circumstance, as described above. This may involve the UPM 180 determining which of the actions if the care plan is appropriate at this time, e.g., based on the sensor data. By way of example, this may involve providing prompts related to coping mechanisms, mindfulness exercises, activities, contacting or accessing assistive resources, etc.

In this exemplary method, the person is then prompted to take/perform the action via a user interface device of the Assistive Device 100, as shown at 620. This may involve the UPM displaying a visual prompt message via a display device 114 of the Assistive Device 100, or providing an audible prompt message via speakers 112a of the Assistive Device 100.

Next, in this exemplary method, a caregiver of the person (e.g., identified in the care plan data) is notified that the person is in a deteriorated state, as shown at 622. This may be performed by the Caregiver Reporting Module 190, e.g., by sending a data communication via the communications network 50 to the caregiver's Caregiver Messaging System 500, and may involve the DAM 170 and/or UPM 180 acting in concert with the CRM 190.

In this exemplary method, method flow returns to 602 to allow for continued sensor data collection and analysis and prompting of the person to avoid a deteriorated state and/or to address a deteriorated state, etc., as shown at 622 and 602.

Accordingly, a person may use an Assistive Device 100 in accordance with the present invention by simply engaging with Assistive Device 100 in a generally conventional manner, e.g., using a PC, smartphone, laptop, etc., while the device gathers data from sensors largely passively, without affirmative action of the person. Additionally, the person may use the Assistive Device by affirmative action/engagement with the device for the purposes described herein—e.g., to provide answers/responses to prompts/questions presented by the Assistive Device, e.g., as part of a health assessment tool for evaluating the person's health. Notably, this may be performed by reading displayed prompts and/hearing spoken/audible prompts, and by responding by touch input, typed input, or spoken input to the Assistive Device. In the case of a mirror or other special-purpose Assistive Device, the persons may be presented with these prompts by engaging in dialog with a computer-generated avatar/persona, in a conversational interaction. Further, the person may be presented with prompts concurrently with an incidentally to use of the Assistive Device for another purpose, e.g., incidentally to use of the mirror device for its primary purpose as a mirror. The person will thereby receive appropriate prompts from the Assistive Device to guide the person to take actions in accordance with the person's personalized care plan that is designed to help the person to avoid a health-deteriorated state, or to address a current health-deteriorated state.

Accordingly, devices and systems in accordance with the present invention may be used to monitor patients' mental, physical, emotional or other health, assess and recognize health states and/or risks, and provide actions to be taken to address or avoid deteriorated/decompensated health states, such as stabilizing coping mechanisms for mental health patients. This is done through multi-faceted data input of an individual's biometric readings including but not limited to facial affect, sleep pattern, blood pressure, heart rate, heart rate variability, from an individual's movement (e.g., GPS data) and activity tracking, from mental health or other screenings via self-administered (with the help of the Assistive Device) health assessments, and/or environmental or other data.

Data collected undergoes analysis to determine whether the person is in a deteriorated state, e.g., by analysis of current data only, or by comparison to generic reference data/norms or to previously-captured specific to the patient's (e.g., as a baseline that was established during the time of hospitalization). A resulting difference indicating a current deteriorated state/current heightened risk of a deteriorated state can result in the device/system assisting the person by prompting the person to take action according to a pre-established care plan, such as a clinician-developed safety plan—e.g., from simply offering coping activities to notifying established Safety Plan contacts/caregivers.

The present invention seeks to improve a person's adherence/compliance with the person's personalized care plan or to maintain a person's wellbeing in an at-home environment—e.g., by adhering to provider visit schedules, performing certain activities, and taking medications. The Assistive Device/system therefore provides effective support to patients in line with their tailored care plans (e.g., safety plans), which is particularly helpful to psychiatric patients during the three initial months following discharge from a psychiatric facility when mental health risks are the highest. Accordingly, the Assistive Devices and system are designed to help users understand all the factors that may lead to crisis/decompensation/deterioration, helping the user to recognize, acknowledge, and react to those factors to prevent decompensation of their mental or other health state with the goal to avoid or mitigate crisis/decompensation/deterioration situations.

The Assistive Device/system also reminds persons of their upcoming visit schedules, activities, coping strategies, and medications from their personalized care plan, e.g., even when the person is not in a deteriorated state.

The Assistive Device/system may automatedly generate/display/transmit or otherwise deliver a message to the person or to a caregiver to initiate an intervention or assistance by the caregiver. For example, this may involve transmitting data to safety plan contacts and/or caregivers for the person.

Alternatively, the Assistive Device/system may automatedly generate/display/transmit or otherwise deliver a message to the person to deliver initiate an intervention to the person. For example, this may involve providing instructions to the person via a user interface device for performance of a coping activity, etc.

In certain embodiments, the Assistive Device/system of the present invention may be used for purposes other than for monitoring for/detecting behavioral health concerns. For example, Assistive Devices using rPPG may be used alone or in combination with data gathered by other devices, e.g., wearable devices, to detect other health concerns, such as minute changes in skin color due to increased blood flow, decreased blood flow, lack of blood flow, etc., which may be associated with heart conditions or other concerns. Similarly, rPPG may be used to detect facial affect associated with health concerns. Facial affect includes a tone of voice, a smile, a frown, a laugh, a smirk, a tear, pressed lips, a crinkled forehead, a scrunched nose, furrowed eyebrows, an eye gaze, or any other facial expression or body movement that indicates emotion.

### OTHER EMBODIMENTS

As referred to above, the present invention is described above, for illustrative purposes only, with reference to an exemplary embodiment for illustrative purposes. It should be noted however that this example is non-limiting, and that the present invention is equally applicable in other contexts.

Additionally, computer readable media storing computer readable code for carrying out the method steps identified above is provided. The computer readable media stores code configured to carry out processes and subprocesses for carrying out the method(s) described herein.

A computer program product recorded on a computer readable medium for carrying out the method steps identified herein is provided. The computer program product comprises computer readable code configured to carry out the method(s) described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A computerized health assistive device for monitoring a person's health and providing care plan-based guidance to the person, the health assistive device comprising:
a processor;
a memory operatively connected to said processor, said memory storing a care plan for the person, said care plan identifying at least one action to be taken by the person in relation to a deteriorated health state;
a user interface device operatively connected to said processor and operable to provide at least one of an audible prompt and a visual prompt to the person;
at least one sensor configured to gather data relevant to assessment of the person's health;
a data analysis module operable to analyze data from said at least one sensor and to determine as a function of said data whether the person is in the deteriorated health state; and
a user prompting module operable to identify a specific action to be taken from said at least one action to be taken in said care plan stored in said memory, and to communicate said specific action to be taken via said user interface device as a prompt to said person.

2. The computerized health assistive device of claim 1, wherein said computerized health assistive device is configured as one of a personal computer, a tablet computer, a laptop computer, a smartphone, and a voice-based assistant device.

3. The computerized health assistive device of claim 1, wherein said computerized health assistive device is configured as a special-purpose assistive device selected from a group consisting of a mirror, a lamp, glass and wearable glasses.

4. The computerized health assistive device of claim 1, wherein said computerized health assistive device is configured as a mirror and comprises:
a housing supporting said processor, said memory, said user interface device, said at least one sensor, said data analysis module, and said user prompting module, and
wherein said at least one sensor comprises a camera supported on said housing and said user interface device comprises a display device defining a first display area configured to display an image of the person captured by said camera.

5. The computerized health assistive device of claim 4, wherein said computerized health assistive device further comprises a second display area configured to display a visual prompt to the person.

6. The computerized health assistive device of claim 5, wherein said computerized health assistive device further comprises a user input device adapted to receive touch input from the person.

7. The computerized health assistive device of claim 5, wherein said computerized health assistive device further comprises a microphone supported on said housing and configured to capture voice responses from the person.

8. The computerized health assistive device of claim 5, wherein said computerized health assistive device further comprises speaker supported on said housing and configured to provide audible prompts to the person and a microphone supported on said housing and configured to capture voice responses from the person, and
wherein said display device defines a third display area configured to display an image of an avatar as animated to appear to speak the audible prompts.

9. The computerized health assistive device of claim 1, wherein said computerized health assistive device comprises:
a housing supporting said processor, said memory, said user interface device, said least one sensor, said data analysis module, and said user prompting module, and
wherein said at least one sensor comprises a speaker supported on said housing and configured to provide audible prompts to the person and a microphone supported on said housing and configured to capture voice responses from the person, and
wherein said user interface device comprises a display device defining a first display area configured to display an image of an avatar as animated to appear to speak the audible prompts.

10. The computerized health assistive device of claim 9, wherein said computerized health assistive device further comprises a second display area configured to display a visual prompt to the person.

11. The computerized health assistive device of claim 10, wherein said computerized health assistive device further comprises a user input device adapted to receive touch input from the person.

12. The computerized health assistive device of claim 5, wherein said computerized health assistive device further comprises wherein said at least one sensor comprises a camera supported on said housing, and wherein said user interface device comprises a display device defining a third display area configured to display an image of the person captured by said camera.

13. The computerized health assistive device of claim 1, wherein said computerized health assistive device is configured as one or a lamp and a mirror and comprises:
a housing supporting said processor, said memory, said user interface device, said least one sensor, said data analysis module, and said user prompting module, and
wherein said at least one sensor comprises a speaker supported on said housing and configured to provide audible prompts to the person and a microphone supported on said housing and configured to capture voice responses from the person.

14. The computerized health assistive device of claim 1, wherein said at least one sensor is integrated into a housing of the computerized health assistive device as a local sensor.

15. The computerized health assistive device of claim 14, wherein said at least one sensor is selected from a group consisting of a remote photoplethysmography sensor, a camera, a microphone, a blood pressure sensor, a heart rate sensor, a heart rate variability sensor, a biometric sensor, a weather sensor, an environmental sensor, a wearable device sensor, and an internet-of-things device sensor.

16. The computerized health assistive device of claim 1, wherein said at least one sensor is separate from a housing of the computerized health assistive device as a remote sensor.

17. The computerized health assistive device of claim 16, wherein said at least one sensor is selected from a group consisting of a remote photoplethysmography sensor, a camera, a microphone, a blood pressure sensor, a heart rate sensor, a heart rate variability sensor, a biometric sensor, a weather sensor, an environmental sensor, a wearable device sensor, and an internet-of-things device sensor.

18. The computerized health assistive device of claim 1, wherein said data analysis module is operable to analyze voice tone from a voice sample captured via a microphone to determine whether the person is in the deteriorated health state.

19. The computerized health assistive device of claim 1, wherein said data analysis module is operable to perform natural language processing to analyze substantive responses from a voice sample captured via a microphone to determine whether the person is in the deteriorated health state.

20. The computerized health assistive device of claim 1, wherein said data analysis module is operable to perform natural language processing to analyze substantive responses from voice sample responses captured via a microphone in response to questions from an assessment tool to determine whether the person is in the deteriorated health state.

21. The computerized health assistive device of claim 1, wherein said data analysis module is operable to compare currently-captured sensor data to previously-captured sensor data to determine whether the person is in the deteriorated health state.

22. The computerized health assistive device of claim 1, wherein said at least one action of said care plan is derived from a clinician's safety plan developed for the person that identifies at least one of a coping mechanism, an activity, a mindfulness exercise and an assistive resource.

23. The computerized health assistive device of claim 1, wherein said at least one action of said care plan is derived from input from the person via the user interface device to self-identify at least one of a preferred coping mechanism, an activity, a mindfulness exercise and an assistive resource.

24. The computerized health assistive device of claim 23, wherein said at least one action of said care plan is derived from input from the person via the user interface device in response to questions provided to the person via said user interface device.

25. The computerized health assistive device of claim 1, further comprising:
a caregiver reporting module configured to transmit data to a computing device of a caregiver of the person when the data analysis module determines that the person is in the deteriorated health state.

26. The computerized health assistive device of claim 1, wherein said at least one action of said care plan is at least one of a preferred coping mechanism, an activity, a mindfulness exercise and an assistive resource, and wherein said user prompting module is operable to communicate said specific action to be taken via said user interface device as a prompt to said person when the data analysis module determines that the person is in the deteriorated health state.

27. The computerized health assistive device of claim 1, wherein said at least one action of said care plan is a reminder of at least one of a clinician visit, an activity, and a medication, and wherein said user prompting module is operable to communicate said specific action to be taken via said user interface device as a prompt to said person when the data analysis module determines that the current health state of the person is not indicative of the deteriorated health state.

28. The computerized health assistive device of claim 1, wherein said at least one action of said care plan is a reminder of at least one of an activity, a coping mechanism, and a mindfulness exercise, and wherein said user prompting module is operable to communicate said specific action to be taken via said user interface device as a prompt to said person as a function of sensor data.

29. The computerized health assistive device of claim 1, wherein said at least one action of said care plan is a reminder of at least one of an activity, a coping mechanism, and a mindfulness exercise, and wherein said user prompting module is operable to communicate said specific action to be taken via said user interface device as a prompt to said person as a function of sensor data associated with one of a current weather condition and a current environmental condition of the person.

30. A computerized health assistive device for monitoring a person's health and providing care plan-based guidance to the person, the health assistive device comprising:
a processor,
a memory operatively connected to said processor, said memory storing a care plan for the person, said care plan identifying at least one action to be taken by the person in relation to a deteriorated health state;
at least one sensor configured to gather data relevant to assessment of the person's health, said at least one sensor comprising a camera configured to capture an image of the person and a microphone configured to capture a voice sample from the person;
a user interface device operatively connected to said processor, aid user interface device comprising:
a speaker configured to provide audible prompts to the person; and
a display device defining a first display area configured to display an image of the person captured by said camera, and a second display area configured to display an image of an avatar as animated to appear to speak the audible prompts;
a data analysis module operable to analyze data from said at least one sensor and to determine as a function of said data whether the person is in the deteriorated health state;
a user prompting module operable to identify a specific action to be taken from said at least one action to be taken in said care plan stored in said memory, and to communicate said specific action to be taken via said user interface device as a prompt to said person; and
a housing supporting said processor, said memory, said user interface device, said at least one sensor, said data analysis module, said speaker, and said user prompting module.

31. The computerized health assistive device of claim 30, wherein said display device further defines a third display area configured to display a visual prompt to the person.

32. The computerized health assistive device of claim 30, wherein said data analysis module is further operable to analyze data from at least one of a remote photoplethysmography sensor, a blood pressure sensor, a heart rate sensor, a heart rate variability sensor, a biometric sensor, a weather sensor, an environmental sensor, a wearable device sensor, and an internet-of-things device sensor to determine as a function of said data whether the person is in the deteriorated health state.

33. The computerized health assistive device of claim 32, wherein said at least one action of said care plan is derived from a clinician's safety plan developed for the person that identifies at least one of a coping mechanism, an activity, a mindfulness exercise and an assistive resource.

34. The computerized health assistive device of claim 30, wherein said at least one action of said care plan is derived from input from the person via the user interface device to self-identify at least one of a preferred coping mechanism, an activity, a mindfulness exercise and an assistive resource.

35. The computerized health assistive device of claim 30, wherein said at least one action of said care plan is at least one of a preferred coping mechanism, an activity, a mindfulness exercise and an assistive resource, and wherein said user prompting module is operable to communicate said specific action to be taken via said user interface device as a prompt to said person when the data analysis module determines that the person is in the deteriorated health state.

36. The computerized health assistive device of claim 30, wherein said at least one action of said care plan is a reminder of at least one of a clinician visit, an activity, and a medication, and wherein said user prompting module is operable to communicate said specific action to be taken via said user interface device as a prompt to said person when the data analysis module determines that the person is not in the deteriorated health state.

37. The computerized health assistive device of claim 30, wherein said at least one action of said care plan is a reminder of at least one of an activity, a coping mechanism, and a mindfulness exercise, and wherein said user prompting module is operable to communicate said specific action to be taken via said user interface device as a prompt to said person as a function of sensor data.

38. The computerized health assistive device of claim 30, wherein said at least one action of said care plan is a reminder of at least one of an activity, a coping mechanism, and a mindfulness exercise, and wherein said user prompting module is operable to communicate said specific action to be taken via said user interface device as a prompt to said person as a function of sensor data associated with one of a current weather condition and a current environmental condition of the person.

39. A computerized health assistive device for monitoring a person's health and providing care plan-based guidance to the person, the health assistive device comprising:
a processor;
a memory operatively connected to said processor, said memory storing a care plan for the person, said care plan identifying at least one action to be taken by the person in relation to a deteriorated health state;
at least one sensor configured to gather data relevant to assessment of the person's health, said at least one sensor comprising a microphone configured to capture a voice sample from the person;
a user interface device operatively connected to said processor, aid user interface device comprising:
a speaker configured to provide audible prompts to the person; and
a data analysis module operable to analyze data from said at least one sensor and to determine as a function of said data whether the person is in the deteriorated health state;
a user prompting module operable to identify a specific action to be taken from said at least one action to be taken in said care plan stored in said memory, and to communicate said specific action to be taken via said user interface device as a prompt to said person; and
a housing supporting said processor, said memory, said user interface device, said at least one sensor, said data analysis module, said speaker, said data analysis module, and said user prompting module.
